(19) United States
(12) Patent Application Publication
(10) Pub. No.: US 2003/0175295 A1
(43) Pub. Date: Sep. 18, 2003

(54) Composition and method for reducing odors from metabolic processes

(76) Inventor: Richard Ticktin, Ft. Lauderdale, FL (US)

Correspondence Address: BOZICEVIC, FIELD & FRANCIS LLP, 200 MIDDLEFIELD RD, SUITE 200, MENLO PARK, CA, 94025, US

(21) Appl. No.: 10/384,401

(22) Filed: Mar. 6, 2003

Related U.S. Application Data
(60) Provisional application No. 60/364,843, filed on Mar. 15, 2002.

(51) Int. Cl. A61K 35/84

(52) U.S. Cl. 424/195.15

(57) ABSTRACT

A sustained release oral dosage formulation and a method of reducing odors resulting from animal waste is disclosed. The composition is comprised of a pharmaceutically acceptable carrier which is preferably a cellulose-based carrier having an odor reducing amount of an active component therein. The active component is preferably Agaricus bisporus extract which is preferably uniformly dispersed in the carrier and provided in a controlled release capsule formulation which can be orally administered.

### CROSS-REFERENCE

This application claims the benefit of U.S. Provisional Application No. 60/364,843, filed Mar. 15, 2002, which application is incorporated herein by reference.

### FIELD OF THE INVENTION

This invention relates to compositions and methods for compositions and methods for reducing odors resulting from metabolic processes, and to processes for manufacturing the same. In particular, the compositions and methods relate to sustained release oral dosages that reduce odors resulting from metabolic processes in animals.

### BACKGROUND OF THE INVENTION

Significant efforts have been made throughout the years to reduce or eliminate fecal, urine and other excrement odors resulting from the metabolic processes in animals, such as household pets and humans.

With recent advancements in quality of life treatments for the elderly or infirmed, the problem of malodorous smells originating from within individual bodies, such as with the putrid stench of feces and urine has become a matter of great concern. Such problems have greatly affected the caregiver/patient relationship, and even personal relations between individuals.

The digestive processes of animals frequently generate sulphur and nitrogen compounds, compounds that contribute to urine and fecal odors. Previous attempts have been made to cover or conceal such odors by spraying an odoriferous animal with or encouraging ingestion of chlorophyll and related compounds. Other proposed solutions have included food additives containing water-soluble copper and iron compounds with a water-soluble phosphate. A portion of the metals present in such additives may be in the form of inedible water-soluble ionized salts, including chlorides. As a result, there have been proposed a variety of liquids for removing or preventing foul odors. However, most of these liquids are mouthwashes, and often contain ingredients undesirable for internal use and therefore use thereof is restricted to the deodorization of mouth.

Additionally, there has been proposed to use compounds that make use of components present in consumables, thereby taking safety into consideration. For instance, the following substances are known to be effective as deodorizers: green tea extract, red beetroot, cacao beans, coffee beans and parsley extracts, perilla extract, persimmon extract, butterburr plant extract, layer, and finely ground material of bracket fungus of the genus Fomes.

Other deodorizers effectively used in the food industry have been proposed as well, including dimethylaminosulfonate, glucosamine, cyclodextrin, organic acids such as 1-ascorbic acid, benzoic acid, gluconic acid, folic acid and nicotinic acid and salts thereof.

However, these compounds fail to prevent the emanation of rancid odors discharged from the inside of the body after foods are ingested. As a result, these methods do not have a satisfactory effect for deodorizing wastes discharged by the animals.

Processes and composition for the treatment of animal waste and the reduction of odor are described in U.S. Pat. No. 6,410,305 issued Jun. 25, 2002. Further, compositions for controlling fecal odor production is described within U.S. Pat. No. 6,344,141 issued Feb. 5, 2002. In addition, various processes for treating animal waste and reducing sulfides and thereby reducing odor are taught within U.S. Pat. No. 5,958,758 issued Sep. 28, 1999. A pet litter box system which prevents the development of unpleasant odors is taught within U.S. Pat. No. 5,031,578 issued Jul. 16, 1991. Further, various deodorant compositions are described within U.S. Pat. No. 6,294,161 issued Sep. 25, 2001. All of these patents are incorporated herein by reference for disclosing and describing particular compositions and methods as they might be used in connection with the methods of reducing odor.

Prior to the present invention, sustained release compositions and methods for reducing odors resulting from metabolic processes that satisfactorily solve the weaknesses associated with the more conventional methods, such as those discussed above, in an efficient, practical and economically affordable manner have not been proposed or suggested by those skilled in the art.

Thus, there is a need for a sustained release composition and method for reducing odors resulting from metabolic processes that overcomes the aforementioned shortcomings of the prior art.

### SUMMARY OF THE INVENTION

The present invention eliminates the above-mentioned needs for a novel sustained release composition and method for reducing odors resulting from metabolic processes in animals, by providing a sustained release oral dosage composition in which there is a therapeutically effective amount of an Agaricus bisporus extract dispersed in a cellulose-based carrier.

In accordance with the present invention, there is provided a sustained release oral dosage composition for reducing odors resulting from metabolic processes in animals comprising a cellulose-based carrier and a therapeutically effective amount of an Agaricus bisporus extract dispersed in the cellulose-based carrier.

The present invention is additionally directed to a method for reducing odors resulting from metabolic processes in animals, the method comprising the steps of providing a cellulose-based carrier with a therapeutic amount of an Agaricus bisporus extract dispersed therein to a recipient, consuming the cellulose-based carrier with the therapeutic amount of the Agaricus bisporus extract dispersed therein by the recipient, and diminishing the amount of at least one odor-forming by-product of the metabolic processes by the therapeutic amount of the Agaricus bisporous extract.

The present invention is further directed to a process for manufacturing a sustained release oral dosage composition for reducing odors resulting from metabolic processes in animals, the process comprising the steps of providing a cellulose-based carrier and adding a therapeutically effective amount of an Agaricus bisporous extract to the cellulose-based carrier.

These and other objects, advantages, and features of the invention will become apparent to those persons skilled in the art upon reading the details of the compositions and methods as more fully described below.

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a carrier” includes a plurality of such carriers and reference to “the composition” includes reference to one or more composition and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In the preferred embodiment of the present invention, a sustained release oral dosage composition for reducing odors resulting from metabolic processes in animals includes a cellulose-based carrier and a therapeutically effective amount of an Agaricus bisporus extract alone or in combination with a gut floral promoter such as Lactobacillus dispersed in the cellulose-based carrier.

Although the active ingredient is preferably Agaricus bisporus, other enzyme containing mushrooms of the Geneva agaricus or Boletus such as Boletus pulverulantus may be used. See also U.S. Pat. No. 6,521,817 issued Feb. 18, 2003 and U.S. Pat. No. 6,343,435 issued Feb. 5, 2002 regarding hybrids of Agaricus bisporus, and these patents are incorporated here to disclosed and describe such.

The cellulose-based carrier of the present invention can be selected from the group consisting of carboxymethylcellulose, hydroxypropyl methylcellulose, and the like. The preferred cellulose-based carrier is hydroxypropyl methylcellulose. The use of a cellulose-based carrier provides for the sustained release of the composition in the animal, thereby prolonging the desired effect of reducing the offensive odors that result from metabolic processes.

Hydroxypropyl methylcellulose having viscosities of between 50 and 4000 centipoise for 2% aqueous solutions at 20° C. are known and commercially available as Methocel E-50 and E-4M, respectively (Dow Chemical Company registered trademarks). The hydroxypropyl methylcellulose that is used as the carrier of the present invention should have an average molecular weight above 40,000, preferably above 200,000. The hydroxypropyl methylcellulose will comprise approximately 199 weight percent of the total weight of the composition.

In one embodiment of the present invention, the dosage form can be a tablet that is prepared by mixing the above-described components with various conventional well-known solvents to form granules. The granules are then compressed into a tablet and an external seal coating is applied. In addition, various diluents, excipients, lubricants, dyes, pigments, dispersants etc. which are disclosed in Remington's Pharmaceutical Sciences, 1995 Edition may be used to optimize the formulations of the invention. In the alternative, dry granulation techniques may be used to prepare the formulation for making compressed tablets. A conventional tabletting machine may be used to compress a granulated mixture of the components of the present invention into a tablet.

The compressed tablets may optionally be coated with a color coat that rapidly disintegrates or dissolves in water or the environment of use, such as the recipient's digestive system. The color coat may be a conventional sugar or polymeric film coating that is applied in a coating pan or by conventional spraying techniques. Preferred materials for the color coat are commercially available under the OPADRY trade name. Generally, the color coat surrounding the core will comprise from about 1 to 5% of the total weight of the tablet.

Additionally, an alternative embodiment of the present invention can be the encapsulation of the composition in a capsule, such as a gel capsule, as is known in the art. Moreover, the composition of the present invention can be ingested as liquid as well.

The cellulose-based carrier base may optionally contain a gut flora promoter. The gut flora promoter of the present invention can include a probiotic dietary fiber, a Lactobacillus species of bacteria, or a Bifidobacterium species of bacteria. The gut flora promoter of the present invention provides further odor reducing effects by facilitating the populating the digestive tract with bacteria that promote less offensive scents.

The preferred embodiment of the present invention further includes an extract of the fungus Agaricus bisporus. Agaricus bisporus is well known to mycophagists as the common “button mushroom” of commerce. Moreover, the ability of Agaricus bisporus to reduce offensive odors is also known.

In practicing the preferred method for reducing odors resulting from metabolic processes in animals, a cellulose-based carrier with a therapeutic amount of an Agaricus bisporus extract dispersed therein is given to a recipient. The recipient then consumes the cellulose-based carrier with the therapeutic amount of the Agaricus bisporus extract, thereby diminishing the amount of at least one odor-forming by-product of the metabolic processes.

The preferred embodiment of the present invention also includes a process for manufacturing the sustained release oral dosage composition. The process includes the steps of providing a cellulose-based carrier and adding a therapeutically effective amount of an Agaricus bisporous extract to the cellulose-based carrier.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A sustained release oral dosage composition for reducing odors resulting from metabolic processes in animals comprising:
a cellulose-based carrier, and
a therapeutically effective amount of an Agaricus bisporus extract dispersed in said cellulose-based carrier.

2. The composition as claimed in claim 1 further comprising a gut flora promoter.

3. The composition as claimed in claim 2 wherein said gut flora promoter is a probiotic dietary fiber.

4. The composition as claimed in claim 2 wherein said gut flora promoter is a Lactobacillus species.

5. The composition as claimed in claim 2 wherein said gut flora promoter is a Bifidobacterium species.

6. The composition as claimed in claim 1 wherein said composition is in tablet form.

7. The composition as claimed in claim 1 wherein said composition is contained in a capsule.

8. The composition as claimed in claim 1 wherein said composition is in liquid form.

9. A method for reducing odors resulting from metabolic processes in animals, said method comprising the steps of:
providing a cellulose-based carrier with a therapeutic amount of an Agaricus bisporus extract dispersed therein to a recipient;
consuming said cellulose-based carrier with said therapeutic amount of said Agaricus bisporus extract dispersed therein by said recipient; and
diminishing the amount of at least one odor-forming by-product of said metabolic processes by said therapeutic amount of said Agaricus bisporous extract.

10. The method as claimed in claim 9 further comprising the step of providing a gut flora promoter.

11. The method as claimed in claim 10 wherein said gut flora promoter is a probiotic dietary fiber.

12. The method as claimed in claim 10 wherein said gut flora promoter is a Lactobacillus species.

13. The method as claimed in claim 10 wherein said gut flora promoter is a Bifidobacterium species.

14. A process for manufacturing a sustained release oral dosage composition for reducing odors resulting from metabolic processes in animals, said process comprising the steps of:
providing a cellulose-based carrier; and
adding a therapeutically effective amount of an Agaricus bisporous extract to said cellulose-based carrier.

15. The process for manufacturing as claimed in claim 14 further comprising the step of providing a gut flora promoter.

16. The process for manufacturing as claimed in claim 15 wherein said gut flora promoter is a probiotic dietary fiber.

17. The process for manufacturing as claimed in claim 15 wherein said gut flora promoter is a Lactobacillus species.

18. The process for manufacturing as claimed in claim 15 wherein said gut flora promoter is a Bifidobacterium species.
